(19) United States
(12) Patent Application Publication
(10) Pub. No.: US 2023/0301945 A1
(43) Pub. Date: Sep. 28, 2023

(54) STABLE PHARMACEUTICAL COMPOSITIONS OF HYDROXYUREA

(71) Applicant: Slayback Pharma LLC, Princeton, NJ (US)

(72) Inventors: Shailendra Mandge, Hyderabad (IN); Venkateshwar Reddy Keesara, Hyderabad (IN); Harish Gunda, Nizamabad (IN); Naga Venkata Durga Prasad Ketha, Hyderabad (IN); Satheesh Balasubramanian ., Hyderabad (IN); Sumitra Ashokkumar Pillai, Hyderabad (IN); Praveen Kumar Subbappa, Princeton, NJ (US)

(73) Assignee: Slayback Pharma LLC, Princeton, NJ (US)

(21) Appl. No.: 18/163,811

(22) Filed: Feb. 2, 2023

(30) Foreign Application Priority Data
Feb. 3, 2022 (IN) 202241005800

(51) Int. Cl. A61K 31/17; A61K 47/18; A61K 9/08; A61K 47/14; A61K 47/26; A61K 47/36; A61K 47/38

(52) U.S. Cl. CPC A61K 31/17; A61K 9/08; A61K 47/14; A61K 47/183; A61K 47/26; A61K 47/36; A61K 47/38

(57) ABSTRACT

The present invention relates to stable oral pharmaceutical compositions in the form of a solution comprising:
hydroxyurea at a concentration of about 10 mg/mL to about 500 mg/mL;
at least one pharmaceutically acceptable liquid vehicle; and
one or more amino acids selected from the group consisting of glycine, alanine, glutamic acid, L-arginine, lysine, L-cysteine, methionine and mixtures thereof, wherein the level of carbamoyloxyurea in the solution is less than 0.5 % w/w as measured by HPLC when the solution is stored at 25° C./60% RH for at least 3 months. The invention further relates to processes for the production of the compositions, methods for their administration, and their use for treatment of diseases treatable by hydroxyurea.

### FIELD OF THE INVENTION

The present invention further relates to stable oral liquid pharmaceutical compositions of hydroxyurea, methods for their administration, processes for their production, and use of these compositions for treatment of diseases treatable by hydroxyurea.

### BACKGROUND OF THE INVENTION

Hydroxyurea, also known as Hydroxycarbamide, is used as an antimetabolite to treat certain types of cancer, as well as to reduce the frequency of pain crises and the need for blood transfusions in patients with sickle cell disease. Hydroxyurea exerts its action through inhibition of DNA synthesis by targeting ribonucleotide reductase enzyme. It is freely soluble in water and is almost completely absorbed from the gastrointestinal tract with a peak plasma level reached within 1 to 4 hours. Chemically, this compound belongs to the class of ureas, and is represented by the structural formula (I):

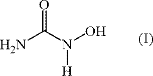

Presently, hydroxyurea is marketed in the United States under brand names Hydrea® (hydroxyurea capsules; 500 mg); Droxia® (hydroxyurea capsules; 200 mg, 300 mg and 400 mg) and Siklos® (hydroxyurea scored-tablets; 100 mg and 1000 mg). Hydrea® is known to be effective in adults, either alone or in conjunction with other antitumor agents or radiation therapy, in the treatment of resistant chronic myeloid leukemia (CML) and locally advanced squamous cell carcinomas of the head and neck (excluding the lip). Droxia® and Siklos®, on the other hand, are indicated to reduce the frequency of painful crises and to reduce the need for blood transfusions in patients with sickle cell anemia with recurrent moderate to severe painful crises. However, use of Droxia® is confined to adult population and Siklos® is proven effective in adult and pediatric population aged 2 years and older.

Although the commercially available solid dosage forms of hydroxyurea are meant for treating adult as well as for pediatric population, they lack in ease-of-use for pediatric population or persons with dysphagia due to several factors, such as the ability to swallow or palatability issues. Likely solution to these problems is to provide an alternate dosage form of hydroxyurea which is advantageous in comparison to solid oral dosage forms of hydroxyurea.

In addition, hydroxyurea is classified as a narrow therapeutic index drug, whereby small changes in the dosage of hydroxyurea can potentially lead to sub-therapeutic or toxic effects. The posology of hydroxyurea is based on the patient’s body weight (kg), wherein the recommended starting dose is 15-20 mg/kg once daily and usual maintenance dose is escalated at 5 mg/kg/day every 8 weeks, based on the blood counts, with a maximum tolerable dose of 35 mg/kg/day. This demonstrates that dosages of hydroxyurea need to be individualized to achieve the best clinical outcomes. It is difficult to individualize doses using solid capsules or tablets. Individual dose requirements of patients and relatively narrow therapeutic index of hydroxyurea mandates the necessity of a suitable dosage form which is sufficiently flexible to allow accurate dosing & enable optimal dose adjustments for individual patient’s needs. The best way to administer a precisely individualized dose of any drug is through the use of an oral liquid solution.

In general practice, hydroxyurea is extemporaneously compounded to prepare aqueous solutions for oral administration from Hydrea® (hydroxyurea capsules). This approach widely benefits pediatric, geriatric, disabled, and incapacitated patients, and persons with dysphagia, who may refuse or find it difficult to take hydroxyurea in solid form due to difficulty in swallowing and / or lack of palatability. However, such manipulated dosage forms fall outside the regulatory agency approval process and compounding results in “off-label” use of medications, with efficacy and safety concerns.

Furthermore, hydroxyurea is a cytotoxic drug and hence it is advisable to administer it only under supervision of qualified clinicians experienced in use of cytotoxic therapy. Caution should be exercised by hospital personnel (i.e., pharmacy technicians, caregivers and nurses), when handling hydroxyurea formulations or when extemporaneously preparing liquid formulations of hydroxyurea for oral administration. Several studies have shown that exposure to cytotoxic drugs can cause reproductive toxic effects as well as carcinogenic effects.

As such hydroxyurea is an ideal candidate for a solution formulation as it is freely soluble in water. However, hydroxyurea has been reported to exhibit poor stability in aqueous solutions due to rapid degradation. This is further supported by the fact that currently there are no commercially available liquid formulations of hydroxyurea in the US. There exists a need for developing ready-to-use, stable liquid compositions of hydroxyurea that are suitable for oral administration and which remain stable over extended period of time under suitable storage conditions.

PCT Application Publication No. WO 2019/220134, discloses stable aqueous solution of hydroxyurea, wherein the pH is adjusted between 6.1 to 7.1 using sodium hydroxide, potassium hydroxide, sodium bicarbonate, and sodium carbonate, or mixtures thereof. Such a narrow pH range had surprising effects in maintaining the stability of the final product with respect to impurity profile. The use of sodium hydroxide as the pH adjuster was tested and found to be stable. It is hypothesized that given the other listed pH adjusters operate in substantially the same manner, the other pH adjusters are also suitable. There remains a need for a stable and robust form of hydroxyurea liquid formulation which can be formulated and maintained at a wider pH range.

Because of the problems associated with commercially approved and manipulated dosage forms of hydroxyurea, it is desirable to develop stable liquid compositions of hydroxyurea suitable for oral administration to human subjects, which are ready-to-use and safe-to-administer, which allow flexibility in administration of doses, which are therapeutically effective, and exhibit prolonged room temperature stability without any significant loss of potency, enabling optimal usage of hydroxyurea compositions.

The present invention fulfils such need by developing stable oral liquid compositions of hydroxyurea to achieve an improved standard of patient care.

### SUMMARY OF THE INVENTION

The present invention relates to stable liquid pharmaceutical compositions of hydroxyurea, or pharmaceutically acceptable salts thereof, suitable for oral administration for treatment of diseases treatable by hydroxyurea. The inventive hydroxyurea compositions are advantageously stable ready-to-use (RTU) or safe-to-administer (STA), suitable for oral administration.

The present invention relates to stable liquid solutions of hydroxyurea, wherein said solution is a palatable solution formulated for oral administration. The present invention also provides liquid pharmaceutical compositions of hydroxyurea having extended stability, while maintaining a potency appropriate for a pharmaceutical dosage form.

In an aspect, the present invention relates to a pharmaceutical composition comprising hydroxyurea at a concentration of about 10 mg/mL to about 500 mg/mL; at least one pharmaceutically acceptable liquid vehicle; and one or more amino acids selected from the group consisting of glycine, alanine, glutamic acid, L-arginine, lysine, L-cysteine, methionine and mixtures thereof, wherein the composition is in the form of a solution suitable for oral administration, and wherein the solution is stable, such that the level of carbamoyloxyurea in the solution is less than 0.5 % w/w as measured by HPLC, when the solution is stored at 25° C./60% RH(Relative Humidity) for at least 3 months or at 2-8° C. for at least 12 months.

In another aspect, the present invention relates to a pharmaceutical composition comprising hydroxyurea at a concentration of about 10 mg/mL to about 500 mg/mL; and at least one pharmaceutically acceptable liquid vehicle, wherein the composition is in the form of a solution suitable for oral administration, wherein the solution is free of sodium hydroxide, potassium hydroxide, sodium bicarbonate, or sodium carbonate as pH adjusters or pH modifiers; and wherein the solution is stable, such that the level of carbamoyloxyurea in the solution is less than 0.5 % w/w as measured by HPLC, when the solution is stored at 25° C./60% RH for at least 3 months or 2-8° C. for at least 12 months.

In yet another aspect, the present invention relates to a pharmaceutical composition comprising, hydroxyurea at a concentration of about 10 mg/mL to about 500 mg/mL; and at least one pharmaceutically acceptable liquid vehicle, wherein the composition is in the form of a solution suitable for oral administration, and wherein the solution resists pH change such that the pH remains within a range of 4.0 to 6.0 or 7.0 to 9.0 when stored at 25° C./60% RH for at least 3 months or at 2-8° C. for at least 12 months.

In some embodiments of the invention, the hydroxyurea is present at a concentration of about 100 mg/mL.

In some embodiments of the invention, the amount of amino acids is in the range of 0.1% to 10.0% based on the total weight of the composition. In other embodiments, the concentration of amino acids is in the range of 0.1 mg/mL to 10.0 mg/mL.

In some embodiments, the compositions of the invention are stable for at least 6 months when stored at 25° C./60% RH (Relative Humidity) such that the level of carbamoyloxyurea in the solution is less than 0.5 % w/w as measured by HPLC, 12.

In some embodiments of the invention, the composition further comprises one or more pharmaceutically acceptable excipients selected from the group comprising stabilizers, solubilizers, pH adjusting agents, pH modifiers, buffering agents, thickening agents, anti-oxidants, chelating agents, preservatives, flavoring agents, sweetening agents, coloring agents and mixtures thereof.

In some embodiments, the compositions of the present invention are free of preservatives.

The pharmaceutically acceptable liquid vehicle may be selected from group consisting of water, glycerin, alcohols, propylene glycol, polyethylene glycol, and mixtures thereof.

In another aspect, a process for preparation of the stable liquid pharmaceutical compositions of the invention is provided, wherein the process comprises dissolving hydroxyurea in an aqueous liquid vehicle along other pharmaceutically acceptable excipients to obtain a stable solution.

Another aspect of the invention relates to methods of treatment for reducing the frequency of painful crises and reducing the need for blood transfusions in patients with sickle cell anemia with recurrent moderate to severe painful crises in pediatric and adult patients, comprising orally administering an effective amount of a stable liquid pharmaceutical composition of the invention.

The inventive pharmaceutical composition of hydroxyurea can be safely administered to a patient by any person with minimal exposure.

By way of non-limiting examples, exemplary combinations applicable to the embodiments described in this application may include any combination with one or more of the elements described above.

### DETAILED DESCRIPTION OF THE INVENTION

Unless defined otherwise, all the technical and scientific terms used herein have the same meanings as commonly known to a person of ordinary skill in the art. In case of conflict, the definitions provided herein will prevail. Unless specified otherwise, all the percentages, portions and ratios in the present invention are on weight basis.

The terms “about” when used along with a numerical variable, generally means the value of the variable and all the values of the variable within a measurement or an experimental error (e.g., 95% confidence interval for the mean) or within a specified value (e.g., ± 10%) within a broader range.

As used herein the term “hydroxyurea” refers to hydroxyurea free base or its pharmaceutically acceptable salts, solvates or hydrates thereof. In principle, any crystalline form or amorphous form of hydroxyurea may be used for manufacturing the inventive pharmaceutical compositions of the present invention. Hydroxyurea in the form of ‘free base’ is the most commercially available form.

The term “pharmaceutically acceptable” substances mean those, which, according to a common medical judgment, are suitable to be in contact with a tissue of a patient without any inappropriate toxicity, irritation, allergic response, etc., have a reasonable balance between advantages and disadvantages, and can be applied to its target use effectively.

The term “pharmaceutically acceptable salt” refers to hydroxyurea salts which are formed with inorganic or organic acids and are pharmaceutically acceptable.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The terms “liquid pharmaceutical composition,” refer to a pharmaceutical composition administered to a patient, including solution or suspension.

The term “ready-to-use,” as used herein, refers to a formulation that does not require constitution or dilution with a prescribed amount of diluent, e.g., purified water or other suitable diluent, before use by the designated route.

The term “safe-to-administer,” as used herein, refers to a formulation which is non-toxic and safe for humans, in the proposed concentrations and total doses used.

The terms “dosage”, “dose unit” or “dose” as used herein means the amount of a pharmaceutical formulation comprising therapeutically active agent(s) administered at a time.

By “effective amount” or “therapeutically effective amount” is meant the amount of a drug sufficient to treat, prevent, or ameliorate a condition in a subject or patient. The effective amount of hydroxyurea or pharmaceutically acceptable salt thereof, may be determined and adjusted by a person of ordinary skill to provide the appropriate amount and dosage regimen, e.g., depending upon manner of administration, the age, body weight, sex, and/or general health of the patient.

Within the context of this invention, the term “solution” refers to a mixture of one or more substances dispersed molecularly (i.e., dissolved) in a dissolving liquid medium or vehicle. The solution is preferably homogeneous, in the sense that API is essentially uniformly distributed and uniformly concentrated in the solution.

The term “solubility” means solubility of hydroxyurea or its pharmaceutically acceptable salts in media such as water, alcohols, oils, surfactants, polyols, buffer, gastrointestinal simulated fluid, gastrointestinal fluid and the like.

The term “subject” refers to an animal, including a human or non-human. The terms patient and subject may be used interchangeably herein.

The terms “stable” and “stability” mean that the evolution of the product with time and/or under specific environmental conditions (i.e., temperature, humidity, etc.) has no significant effects on its quality, safety and/or efficacy for a given time period. It can be measured through the formation of degradation products (impurities), variation of pH, microbial growth, or physical appearance, such as precipitation and/or color.

The term “any person” refers to any human being capable of administering dose of hydroxyurea composition to a patient, wherein human being includes physicians, healthcare professions, nurse, pharmacist, pharmacy technicians and the patient.

The term “exposure” refers to accidental contact of hydroxyurea composition to the skin of any person while administering hydroxyurea composition to the patient.

As used herein, “prolonged duration” refers to the holding of a composition under controlled or uncontrolled conditions for a period of more than 30 days.

As used herein, “significant loss of potency” can mean more than about a 10% loss of hydroxyurea under typical commercial storage conditions.

As used herein, “to treat” a condition or “treatment” of the condition is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable.

The present application relates to stable liquid pharmaceutical compositions of hydroxyurea, wherein hydroxyurea is present at a concentration of 10 mg/mL or more.

The pharmaceutical compositions may be formulated according to conventional pharmaceutical practice.

In some embodiments, the pharmaceutical compositions of the invention have a concentration of hydroxyurea of about 10 mg/mL, about 20 mg/mL, about 30 mg/mL, about 40 mg/mL, about 50 mg/mL, about 75 mg/mL, about 100 mg/mL, about 150 mg/mL, 200 mg/mL, about 250 mg/mL, about 300 mg/mL, about 350 mg/mL, about 400 mg/mL, about 450 mg/mL, about 500 mg/mL, or preferably about 100 mg/mL.

Another embodiment provides a method for reducing the frequency of painful crises and to reduce the need for blood transfusions in patients with sickle cell anemia with recurrent moderate to severe painful crises in pediatric and adult patients, by orally administering to a subject from about 10 mg/mL to about 500 mg/mL of the stable liquid pharmaceutical composition of the invention.

In an embodiment the present invention provides a pharmaceutical composition comprising hydroxyurea at a concentration of about 100 mg/mL, at least one pharmaceutically acceptable liquid vehicle; and one or more amino acids selected from the group consisting of glycine, alanine, glutamic acid, L-arginine, lysine, L-cysteine, methionine and mixtures thereof, wherein the composition is in the form of a solution suitable for oral administration and wherein the amount of amino acids is in the range of 0.1% to 10.0% based on the total weight of the composition.

The terms “liquid vehicle” or “pharmaceutically acceptable liquid vehicle” or “solvent” or “pharmaceutically acceptable solvent” as used herein, is any liquid medium used for dilution or dissolution of parenteral, oral or peroral formulations, such as water, aqueous organic solvent, non-aqueous organic solvent and other liquids described herein or used in the pharmaceutical and/or food industry. The liquid vehicle of the present invention may be selected from the group consisting of water, glycerin, alcohols, propylene glycol, polyethylene glycol, and mixtures thereof. Preferably purified water is used as a liquid vehicle. In a preferred embodiment, the inventive composition comprises at least 90% purified water, preferably at least 95% purified water and more preferably at least 99% purified water with respect to total amount of the composition.

In another embodiment, the present invention provides stable liquid pharmaceutical compositions suitable for oral administration comprising (i) hydroxyurea at a concentration of about 100 mg/mL; (ii) at least one pharmaceutically acceptable liquid vehicle; and (iii) optionally one or more pharmaceutically acceptable excipients selected from group comprising of stabilizers, solubilizers, pH modifiers, pH adjusting agents, buffering agents, thickening agents, anti-oxidants, chelating agents, preservatives, flavoring agents, sweetening agents, coloring agents and mixtures thereof.

In one embodiment, the present invention provides stable oral solution, wherein the solution comprises (a) hydroxyurea at a concentration of about 100 mg/mL; (b) at least one pharmaceutically acceptable liquid vehicle; and (c) one or more other pharmaceutically acceptable excipients selected from group comprising of stabilizers, solubilizers, pH modifiers, pH adjusting agents, buffering agents, preservatives, thickening agents, anti-oxidants, chelating agents, flavouring agents, sweetening agents, colouring agents and mixtures thereof, wherein the solution resists pH change such that the pH remains within a range of 4.0 to 9.0 when stored at 25° C./60% RH for at least 3 months or at 2-8° C. for at least 12 months.

In another embodiment, the present invention provides a stable pharmaceutical composition comprising, hydroxyurea at a concentration of about 100 mg/mL; and at least one pharmaceutically acceptable liquid vehicle, wherein the composition is in the form of a solution suitable for oral administration, and wherein the solution resists pH change such that the pH remains within a range of 4.0 to 6.0 when stored at 25° C./60% RH for at least 3 months or at 2-8° C. for at least 12 months.

In yet another embodiment, the present invention provides a stable pharmaceutical composition comprising, hydroxyurea at a concentration of about 100 mg/mL; and at least one pharmaceutically acceptable liquid vehicle, wherein the composition is in the form of a solution suitable for oral administration, and wherein the solution resists pH change such that the pH remains within a range of 7.0 to 9.0 when stored at 25° C./60% RH for at least 3 months or at 2-8° C. for at least 12 months.

In one embodiment, the present invention provides stable oral solution comprising hydroxyurea at a concentration of about 100 mg/mL; and at least one pharmaceutically acceptable liquid vehicle, wherein the composition is in the form of a solution suitable for oral administration, wherein the solution is free of sodium hydroxide, potassium hydroxide, sodium bicarbonate, or sodium carbonate as pH adjusters or pH modifiers; and wherein the solution is stable, such that the level of carbamoyloxyurea in the solution is less than 0.5 % w/w as measured by HPLC, when the solution is stored at 25° C./60% RH for at least 3 months or 2-8° C. for at least 12 months.

The pharmaceutical composition of the present invention may contain a “stability enhancing agent” or “stabilizer”. The terms “stability enhancing agent” or “stabilizer” as used herein inhibits, prevents, slows down, or reduces the degradation of hydroxyurea. More specifically, stability enhancing agents include amino acids, salts, ethylenediaminetetraacetic acid (EDTA), metal ions, gums, celluloses, cyclodextrins, sugars, sugar alcohols, monosaccharides, disaccharides or polysaccharides and combinations thereof. In an embodiment, concentration of the stabilizer ranges from 0.001% to 20%, preferably 0.01% to 15.0%, more preferably 0.1% to 10.0%, based on the total weight of the composition.

Amino acid stabilizers include glycine, alanine, glutamate, sodium glutamate, L-arginine, lysine, L-cysteine or methionine and mixtures thereof. In some embodiments, the amino acid stabilizers are present at a concentrion of 0.1 mg/mL to 10 mg/mL, such as about 0.3 mg/mL to about 8 mg/mL or about 0.5 mg/mL to about 7.5 mg/mL, or about 1.5 mg/mL to about 6 mg/mL, or about 5 mg/mL to about 7 mg/mL.

Salt stabilizers include sodium chloride, sodium sulfate, and mixtures thereof.

Metal ion stabilizers include zinc, magnesium and calcium ions and mixtures thereof.

Gum stabilizers include natural and synthetic gums, such as xanthan gum, carrageenan, guar gum, locust bean gum, pectin, gellan gum, gelatin, gum Arabic, gum karay, gum tragacanth, gum ghatti, agar, konjac, alginate, tara gum, pullulan, curdlan, chitosan, carboxymethylcellulose gum, cellulose, microcrystalline cellulose, methylcellulose, hydroxypropyl methylcellulose, and mixtures thereof.

Cellulose derivatives include, but are not limited to, carboxy methyl cellulose, ethyl cellulose, hydroxyethyl cellulose, hydroxyl propyl cellulose, hydroxyl propyl methylcellulose, methyl cellulose, polyanionic cellulose, and combinations thereof. Different grades of each cellulosic compound or stabilizing agent, corresponding to variations in, e.g., molecular weight, viscosity, solubility, and hydration, are also encompassed by the terms.

Cyclodextrin stabilizer include cyclodextrin, α-cyclodextrin, β-cyclodextrin, δ-cyclodextrin, Y-cyclodextrin and derivatives thereof.

In some embodiments, the stabilizer is a sugar selected from the group consisting of sucrose, mannitol or trehalose and mixtures thereof.

In another embodiment, the stabilizer can be selected from monosaccharides such as glucose, galactose, fructose, or mannose; disaccharides such as sucrose, maltose, or lactose; polysaccharides such as oligosaccharides, starch, cellulose, or mixtures thereof.

In yet another embodiment, the stabilizer is a sugar alcohol selected from erythritol, threitol, arabitol, xylitol, ribitol, mannitol, sorbitol, dulcitol, iditol, isomalt, maltitol, lactitol, polyglycitol or combinations thereof.

In some preferred embodiments, the stabilizer is selected from the group consisting of glycine, L-arginine, L-cysteine, xanthan gum or sodium carboxymethyl cellulose and mixtures thereof.

In some embodiments, the pH of the inventive composition ranges from about 4 to about 9. In some embodiments, the pH is preferably between 4.0 to 6.0. In some embodiments, the pH is preferably between 7.0 to 9.0. The pH of the composition may be between 5.0 to 8.0, or between 6.0 to 7.0, or between 4.0 to 5.0, or between 5.0 to 6.0, or between 7.0 to 8.0, or 7.2 to 9. To provide the selected pH, the composition may optionally include one or more “pH adjusting agents” or “pH adjusters” selected from organic and inorganic acids and bases.

In some embodiments, the inventive composition has resistance to pH change such that the composition has a pH that remains within a range of 4.0 to 9.0, or 4.0 to 6.0, or 7.0 to 9.0, or 5.0 to 8.0, or 6.0 to 7.0, or 4.0 to 5.0, or 5.0 to 6.0, or 7.0 to 8.0, or 7.2 to 9 when stored at 25° C./60% RH for at least 3 months or at 2-8° C. for at least 12 months.

The pH of the composition can be adjusted with any combination of acidic and/or basic pH adjusting agents known in the art. Acidic materials include organic acids and inorganic acids, in particular, monocarboxylic acids, dicarboxylic acids, and tricarboxylic acids, for example, acetic acid, citric acid, tartaric acid, alpha-hydroxy acids, beta-hydroxy acids, salicylic acid, lactic acid, malic acid, glycolic acid, amino acids and natural fruit acids, or inorganic acids, for example, hydrochloric acid, nitric acid, sulfuric acid, sulfamic acid, phosphoric acid, and combinations thereof. Basic materials include inorganic and organic bases, and combinations thereof. Basic pH adjusting agents include organic and inorganic bases. Examples of inorganic bases include alkali metal salts such as sodium borate (borax), sodium phosphate, sodium pyrophosphate, and the like; and mixtures thereof. Examples of organic bases include triethanolamine (TEA), diisopropanolamine, triisopropanolamine, aminomethyl propanol, dodecylamine, cocamine, oleamine, morpholine, triamylamine, triethylamine, tetrakis(hydroxypropyl)ethylenediamine, L-arginine, aminomethyl propanol, tromethamine (2-amino 2-hydroxymethyl-1,3-propanediol), PEG-15 cocamine, and mixtures thereof. Such pH adjusters may be present at a concentration that ranges from 0.001% to 20%, preferably 0.01% to 15.0%, more preferably 0.1% to 10.0%, based on the total weight of the composition.

The inventive compositions may comprise a pH modifier in an amount that sufficiently and effectively regulates the pH of the composition to maintain it within a range from about 4 to about 9. In some embodiments, the pH is preferably between 4.0 to 6.0. In some embodiments, the pH is preferably between 7.0 to 9.0. The pH of the composition may be between 5.0 to 8.0, or between 6.0 to 7.0, or between 4.0 to 5.0, or between 5.0 to 6.0, or between 7.0 to 8.0, or 7.2 to 9. Suitable pH modifiers for use herein include, but are not limited to ammonia; alkanolamines such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3, -propandiol; amino acids such as glycine, alanine, glutamic acid, L-arginine, lysine, L-cysteine or methionine and mixtures thereof; and acidifying agents such as inorganic and inorganic acids, such as phosphoric acid, acetic acid, ascorbic acid, citric acid, tartaric acid, hydrochloric acid, and mixtures thereof. Such pH modifiers may be present at a concentration that ranges from 0.001% to 20%, preferably 0.01% to 15.0%, more preferably 0.1% to 10.0%, based on the total weight of the composition. In most preferred embodiment, the pH modifier is selected from the group comprising of glycine, L-arginine, L-cysteine, or tartaric acid and mixtures thereof.

In some embodiments, the pH modifiers are present at a concentrion of 0.1 mg/mL to 10 mg/mL, such as about 0.3 mg/mL to about 8 mg/mL or about 0.5 mg/mL to about 7.5 mg/mL, or about 1.5 mg/mL to about 6 mg/mL, or about 5 mg/mL to about 7 mg/mL.

An embodiment of the present invention covers a stable oral solution comprising (i) hydroxyurea at a concentration of about 100 mg/mL; (ii) at least one pharmaceutically acceptable liquid vehicle; (iii) one or more other pharmaceutically acceptable excipients, wherein pH of the solution is less than 6.4; and wherein level of carbamoyloxyurea in the solution is less than 0.5 % w/w as measured by HPLC, when said solution is stored at 25° C./60% RH for at least 3 months or at 2-8° C. for at least 12 months.

An embodiment of the present invention covers a stable oral solution comprising (i) hydroxyurea at a concentration of about 100 mg/mL; (ii) at least one pharmaceutically acceptable liquid vehicle; (iii) one or more other pharmaceutically acceptable excipients, wherein pH of the solution is more than 6.8; and wherein level of carbamoyloxyurea in the solution is less than 0.5 % w/w as measured by HPLC, when said solution is stored at 25° C./60% RH for at least 3 months or at 2-8° C. for at least 12 months.

The pharmaceutical compositions of the present invention may optionally contain a buffer. The term “buffer” or “buffering agents” as used herein, is an agent used to resist change in pH upon dilution or addition of acid or alkali. Such compounds include, by way of example and without limitation, sodium dihydrogen phosphate monohydrate, disodium hydrogen phosphate anhydrous, citric acid, ascorbic acid, acetic acid, sodium acetate, adipic acid, benzoic acid, sodium benzoate, sodium citrate, monobasic sodium phosphate, dibasic sodium phosphate, disodium hydrogen phosphate dodecahydrate, lactic acid, tris buffer, tartaric acid, potassium metaphosphate, potassium phosphate, monobasic sodium acetate, sodium ascorbate anhydrous, sodium ascorbate monohydrate, sodium tartrate and others known to those of ordinary skill in the art. In some embodiments, the concentration of buffer in the present invention ranges from 0.001% to 10%, preferably 0.01% to 5.0%, more preferably 0.05% to 1.0%, based on the total weight of the composition.

In addition to stabilizing pharmaceutical preparations against chemical and physical degradation, liquid preparations, especially multi-dose preparations, must usually be protected from microbial contamination. In one embodiment, pharmaceutical composition of the present invention comprise a preservative selected from the group consisting of benzoic acid, sodium or potassium salts thereof, ethanol, isopropanol, methanol, butyl alcohol, benzalkonium chloride, benzethonium chloride, benzyl alcohol, butylparaben, cetylpyridinium chloride, chlorobutanol, chlorocresol, cresol, dehydroacetic acid, ethylparaben, ethylparaben sodium, methylparaben, methylparaben sodium, phenol, phenylethyl alcohol, phenylmercuric acetate, phenylmercuric nitrate, potassium benzoate, potassium sorbate, propylparaben, propylparaben sodium, sodium dehydroacetate, sodium propionate, sorbic acid, thimerosal, thymol, and combinations thereof. In a preferred embodiment, the concentration of preservative ranges from 0.001% to 10%, preferably 0.01% to 5.0%, more preferably 0.05% to 1.0%, based on the total weight of the composition.

The pharmaceutical compositions of the present invention are optionally preservative-free compositions. The term “preservative-free” means that the present compositions and methods comprise no use of preservatives. Hydroxyurea is reported to have a high level of antimicrobial activity on its own. Thus, the need for a preservative in the present pharmaceutical composition is minimal.

As used herein, “chelating agent” refers to an agent which forms via two or more of its functional groups stable complexes with metal ions. Preferably, the chelator is selected from the group consisting of disodium ethylenediaminetetraacetic acid (disodium EDTA), diethylenetriaminepentaacetic acid (DTPA), ethylene glycol-bis (β-amin oethyl ether)-tetra acetic acid (EGTA), N-(hydroxyethyl) ethylenediaminetriacetic acid (HEDTA), nitrilotriacetic acid (NTA), triethanolamine, 8-hydroxyquinoline, gluconic acid, saccharic acid, thiodipropionic acid, acetonic dicarboxylic acid, lecithin, di(hydroxyethyl)glycine, phenylalanine, tryptophan, sorbitol and pharmaceutically acceptable salts and mixtures thereof. More preferably, the chelating agent is selected from the group consisting of disodium EDTA, DTPA, gluconic acid and a pharmaceutically acceptable salts and mixtures thereof. The amount of chelating agent may range from about 0.01 mg/mL to about 1 mg/mL of the composition. In an embodiment, the chelating agent concentration ranges from about 0.001% to about 5% w/v of total composition.

The term “sweetening agents” refers to both bulk (caloric) and intense (non-caloric) sweeteners, which impart sweet taste to the preparation. Examples of bulk sweeteners are dextrose, fructose, glucose, hydrogenated glucose syrup, isomalt, maltitol, maltose, mannitol, sorbitol, sucrose, xylitol, ribose, deoxyribose, neuraminic acid and mixtures thereof. Examples of intense sweeteners are acesulfame, alitame, aspartame, cyclamate, dihydrochalcone sweetener, monellin, neohesperidin, neotame, saccharin, stevioside, sucralose, pharmaceutically acceptable salts thereof, such as sodium or calcium saccharin, acesulfame potassium or sodium cyclamate, and mixtures thereof. In one embodiment, the pharmaceutically acceptable sweetener in the present invention is sucralose. In a preferred embodiment, the concentration of sweetener ranges from 0.001% to 10%, preferably 0.01% to 5.0%, more preferably 0.05% to 1.0%, based on the total weight of the composition.

The term “flavoring agent,” as used herein, refers to an agent or a mixture of agents that adds flavor to a mixture. Flavoring agents include natural flavors, artificial flavors, and mixtures thereof. Flavoring agents include, but are not limited to, mint, peppermint, cola, apple, vanilla, orange, peach, apricot, raspberry, cherry, honey, lemon, coconut, pineapple, strawberry banana, mixed berry, mixed red fruit and cream flavors and mixture thereof. In some embodiments, the flavoring agent is mixed berry flavor. The concentration of flavoring agent ranges from 0.001% to 10%, preferably 0.01% to 5.0%, more preferably 0.05% to 1.0%, based on the total weight of the composition.

In order to thicken the liquid composition and to improve the mouth-feel of the composition, and/or to help coat the lining of the gastrointestinal tract, thickening agents can be optionally included into the present pharmaceutical composition. Thickening agenst may be selected from but not limited to acacia, alginic acid bentonite, carbomer, carboxymethylcellulose calcium or sodium, cetostearyl alcohol, methyl cellulose, ethylcellulose, gelatin guar gum, hydroxyethylcellulose (HEC), hydroxypropylcellulose (HPC), hydroxypropylmethylcellulose (HPMC), maltodextrin, polyvinyl alcohol, povidone, propylene carbonate, propylene glycol alginate, sodium alginate, sodium starch glycolate, starch tragacanth, and xanthan gum, or any combination thereof. In some embodiments, the concentration of the thickening agent ranges from 0.001% to 20%, preferably 0.01% to 15.0%, more preferably 0.1% to 10.0%, based on the total weight of the composition.

In an embodiment, the present invention provides a process for preparation of a stable, liquid pharmaceutical formulation of hydroxyurea for oral administration, wherein the process comprises a) adding preservative, sweetener and/or flavoring agent in a pharmaceutically acceptable solvent, stirring the solution and then adding hydroxyurea under continuous stirring until clear solution is obtained; b) optionally, adding any other pharmaceutically acceptable excipient and stirring until clear solution is obtained c) finally making up the volume of solution to the desired volume, and concentration and filtration of the final clear solution through 5µ PVDF membrane filter and d) filling in a suitable pharmaceutically acceptable container.

In an embodiment, the pharmaceutical composition of present application is filled into suitable pharmaceutically acceptable container, wherein the pharmaceutically acceptable container is selected from the group consisting of bottle, infusion bag, vial, prefilled syringe, syringe and ampoule.

In an embodiment, the pharmaceutically acceptable container is a bottle, wherein the bottle is selected from group consisting of glass bottle or plastic bottle, wherein glass bottle is selected from group consisting of Type I, II and III borosilicate glass bottles, wherein the glass bottle may be amber color glass bottle or clear glass bottle.

In another embodiment, the pharmaceutically acceptable container is a bottle, wherein the bottle is selected from the group consisting of high density polyethylene (HDPE) bottle, polyethylene terephthalate (PET) and polypropylene (PP), wherein the plastic bottle may be amber color, white opaque or translucent plastic bottle.

The glass and HDPE bottles are available in 30, 60, 100, 120, 150, 250 & 500 mL fill volumes, among others.

In an embodiment, the pharmaceutical composition of present application are packed in a kit comprising a bottle with child resistant cap, adapter and dosing syringe.

Stability: As used herein, the term “stable” is defined as no more than about 10% loss of hydroxyurea under typical commercial storage conditions. In certain embodiments of the present invention, the degradation related loss of hydroxyurea is no more than about 5%, no more than about 4%, no more than about 3% loss, no more than about 2% loss, no more than about 1% loss, under typical commercial storage conditions. The composition of the invention retains at least about 90% of the potency of hydroxyurea after storing the composition at 25° C./60%RH condition for at least 6 months. In certain embodiments, the solution retains at least about 95% of the potency of hydroxyurea after storing the solution at 2-8° C. for at least twelve months.

In some embodiments, the present invention provides stable oral solution which are stable for at least 3 months or at least 6 months, or at least 9 months, or at least 12 months, at 25° C./60% RH storage conditions.

In some embodiments, the present invention provides stable oral solutions which are stable for at least 6 months, or at least 9 months or at least 12 months or at least 18 months or at least 24 months, at 2-8° C. storage conditions.

Hydroxyurea has known as well as unknown impurities as part of production or storage degration. In particular, urea, hydroxylamine, and carbamoyloxyurea impurities were monitored.

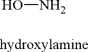

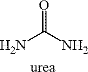

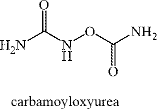

In an embodiment, the present invention provides stable oral solution, wherein the level of total impurities in the composition is less than about 5%w/w, preferably less than about 3%w/w, more preferably less than about 1%w/w, more preferably less than about 0.5%w/w as measured by HPLC.

In another embodiment, the level of any unknown impurity in the inventive pharmaceutical composition resulting from the degradation of hydroxyurea is less than about 5% (w/w), preferably less than about 3% (w/w), preferably less than about 1% (w/w), preferably less than about 0.5% (w/w), preferably less than about 0.15% (w/w) and more preferably less than about 0.1% (w/w) as measured by HPLC.

In yet another embodiment, the level of hydroxylamine impurity or urea impurity or carbamoyloxyurea impurity or any other known impurity in the inventive pharmaceutical composition resulting from the degradation of hydroxyurea is less than about 5% (w/w), preferably less than about 3% (w/w), preferably less than about 1% (w/w), preferably less than about 0.5% (w/w), preferably less than about 0.15% (w/w) and more preferably less than about 0.1% (w/w) as measured by HPLC.

The International Conference on Harmonization of Technical Requirements for Registration of Pharmaceuticals for Human Use (ICH) stipulates that the maximum allowable limit for any degradant in a formulation should not exceed 0.15% w/v, at therapeutic doses of hydroxyurea in the treatment of sickle cell disease. However, rat toxicology studies showed that carbamoyloxyurea does not pose a risk over and above hydroxycarbamide (hydroxyurea) as disclosed in PCT Application Publication No. WO 2019/220134. As a result, the European Medicines Agency has now accepted that carbamoyloxyurea contents of up to 0.5% w/v are acceptable. Id.

In light of the above, in the context of the present invention, stable formulations may have up to 0.5% w/v carbamoyloxyurea after storage for the relevant period within the necessary temperature range. A formulation will not be deemed to be stable if the carbamoyloxyurea content exceeds 0.5% w/v when stored at the necessary temperature range. That is, in order to be stable, the carbamoyloxyurea content in the pharmaceutical composition cannot exceed 0.5% w/v after storage for the relevant period within the necessary temperature range.

Dosage and administration: In one embodiment, the dose of hydroxyurea is in the range of from about 0.1 mg/kg/day to about 100 mg/kg/day, preferably in the range from about 0.5 mg/kg/day to about 80 mg/kg/day and more preferably in the range from about 1.0 mg/kg/day to about 60 mg/kg/day

The dosage levels can be dependent on the nature of the condition, drug efficacy, the condition of the patient, the judgment of the practitioner, and the frequency and mode of administration. The unit dosage forms can be administered to achieve any daily amount described herein, such as by administering one to five times daily (e.g., one, two, three, four, or five times daily).

HPLC procedure: As explained in detail below, the following HPLC procedure was used to detect, quantify impurities of hydroxyurea and to determine assay % of hydroxyurea and preservative. The materials and methods are listed below:

Assay of hydroxyurea

| Chromatographic Mode | HPLC system equipped with UV/PDA detector | | |
|---|---|---|---|
| Column | L1, 250 × 4.6 mm, 5 µm | | |
| Wavelength | 214 nm | | |
| Flow rate | 0.5 mL/min | | |
| Injection volume | 10 µL | | |
| Column temperature Temperature | 25° C. | | |
| Sample temperature | 5° C. | | |
| Run time | 30 Minutes | | |
| Mobile Phase A | pH 5.0, 10 mm Phosphate Buffer contains 1.74 g of tetra butyl ammonium hydrogen sulfate per 1000 mL. | | |
| Mobile Phase B | Methanol | | |
| Mode of Elution | Gradient | | |
| | Time | Mobile Phase-A (%) | Mobile Phase-B (%) |
| | 0 | 85 | 15 |
| | 8 | 65 | 15 |
| | 10 | 2 | 98 |
| | 20 | 2 | 98 |
| | 21 | 85 | 15 |
| | 30 | 85 | 15 |

Assay of Methylparaben

| | |
|---|---|
| Chromatographic Mode | HPLC system equipped with UV/PDA detector |
| Column | L1, 250 × 4.6 mm, 5 µm |
| Wavelength | 254 nm |
| Flow rate | 1.0 mL/min |
| Injection volume | 20 µL |
| Column temperature Temperature | 30° C. |
| Sample temperature | 5° C. |
| Run time | 16 Minutes |
| Buffer | pH 4.0, 30 mm sodium acetate Buffer. |
| Mobile Phase | Buffer: Methanol 40:60 (v/v) |
| Mode of Elution | Isocratic |

Related Substances

| Chromatographic Mode | HPLC system equipped with UV/PDA detector | | |
|---|---|---|---|
| Column | Cosmosil Hilic 250×4.6 mm, 5 µ | | |
| Wavelength | 206 nm | | |
| Flow rate | 0.5 mL/min. | | |
| Injection volume | 10 µL | | |
| Column temperature Temperature | 30° C. | | |
| Sample temperature | 5° | | |
| Run time | Blank, Samples - 65 minutes (Gradient) Standard - 30 Minutes (Isocratic) | | |
| Mobile Phase A | Water and Acetonitrile in the ratio of 5:95 V/V. | | |
| Mobile Phase B | Water and Acetonitrile in the ratio of 30:70 V/V | | |
| Mode of Elution | Gradient | | |
| | Time | Mobile Phase-A (%) | Mobile Phase-B (%) |
| | 0 | 100 | 0 |
| | 30 | 100 | 0 |
| | 40 | 0 | 100 |
| | 45 | 0 | 100 |
| | 46 | 100 | 0 |
| | 65 | 100 | 0 |

### EXAMPLES

The following examples are exemplary and not intended to be limiting. The above disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

### Example 1

Hydroxyurea compositions are set forth in Table 1

TABLE 1

| Ingredients | Composition (mg/mL) | |
|---|---|---|
| | A | B |
| Hydroxyurea | 100.0 | 100.0 |
| L-Arginine | 0.5 | - |
| Glycine | 5.7 | 5.7 |
| L-Cysteine HCl monohydrate | - | 0.3 |
| Methyl paraben | 0.5 | - |
| Disodium EDTA | - | 2.5 |
| Sucralose | 2.0 | 2.0 |
| Mixed berry flavor | 0.5 | 0.5 |
| Purified water | q.s | q.s |
| Final pH | 7.53 | 4.54 |

### Manufacturing Procedure of Compositions A and B:

Methyl paraben, sucralose and mixed berry flavor was added to purified water (70% of the total quantity) and stirred continuously to get a clear solution. (For composition A, purified water was heated at 80° C. to dissolve preservative. The solution was later cooled to room temperature and sweetener, followed by flavor was added and stirred to get a clear solution). Hydroxyurea was added to the solution under continuous stirring at room temperature to get a clear solution. Further, L-Arginine, L-cysteine and Glycine and disodium EDTA were added to above solution and stirred continuously at room temperature to get a clear solution. Purified water was added to make up the final volume. The solution was filtered using 5µ Polyvinylidene fluoride (PVDF) membrane filter and final filtered solution was filled in Amber Type I glass bottles or HDPE containers. Stability of composition A and B was evaluated at 2-8° C. and at 25° C./60%RH conditions.

TABLE 2

Stability data for Composition A

| Stability condition and Pack | Initial | 25° C./60% RH-HDPE bottle | 2-8° C.-HDPE bottle |
|---|---|---|---|
| Station | | 3 M | 12 M |
| Description | Clear | Clear | Clear |
| pH | 7.53 | 6.69 | 7.06 |
| Assay | 99 | 98.2 | 102.4 |
| Preservative content | 100.1 | 99.5 | 99.7 |
| Related Substances | | | |
| carbamoyloxyurea | ND* | -- | 0.06 |
| Highest Unknown | 0.20 | 0.81 | 0.07 |
| Total impurities | 0.24 | 1.91 | 0.18 |

*ND = Not Detected

TABLE 3

Stability data for Composition B

| Stability condition and Pack | Initial | 25° C./60% RH- HDPE bottle | 2-8° C.-HDPE bottle |
|---|---|---|---|
| Station | | 6 M | 12 M |
| Description | Clear | Clear | Clear |
| pH | 4.54 | 6.18 | 5.8 |
| Assay | 100.7 | 95.7 | 99.0 |
| Related Substances | | | |
| carbamoyloxyurea | ND* | 0.09 | 0.01 |
| Highest Unknown | 0.05 | 0.25 | 0.25 |
| Total impurities | 0.07 | 0.40 | 0.26 |

*ND = Not Detected

The Composition A and B are physically and chemically stable for at least 3 months at 25° C./60% RH and for at least 12 months at 2-8° C., without visible particles & with no significant change in assay.

### Example 2

TABLE 4

| Ingredients | Composition (mg/mL) | | |
|---|---|---|---|
| | C | D | E |
| Hydroxyurea | 100.0 | 100.0 | 100.0 |
| L-Arginine | 0.5 | 0.5 | 0.5 |
| Glycine | 5.7 | -- | -- |
| Methyl paraben | 0.5 | 0.5 | 0.5 |
| Sucralose | 2.0 | 2.0 | 2.0 |
| Xanthan gum | 4.0 | -- | -- |
| Sodium carboxymethylcellulose | -- | 2.5 | 5.0 |
| Mixed berry flavor | 0.5 | 0.5 | 0.5 |
| Purified water | q.s | q.s | q.s |
| Final pH | 7.66 | 7.83 | 7.96 |

### Manufacturing Procedure of Compositions C, D and E:

Purified water (70% of the total quantity) was heated at 80° C. and methyl paraben was added and stirred continuously to get a clear solution. The solution was cooled to room temperature and sweetener, followed by flavor was added and stirred to get a clear solution. Hydroxyurea was added to the solution under continuous stirring at room temperature to get a clear solution. Further, respective stabilizers and pH modifiers were added to the above solution and stirred continuously at room temperature to get a clear solution. Purified water was added to make up the final volume. The solution was filtered using 5µ PVDF membrane filter and final filtered solution was filled in Amber Type I glass bottles or HDPE containers. Stability of composition C was evaluated at 2-8° C. and at 25° C./60%RH conditions.

TABLE 5

Stability data for Composition C

| Stability condition and Pack | Initial | 25° C./60% RH HDPE bottle | | 2-8° C. HDPE bottle | |
|---|---|---|---|---|---|
| Station | | 1 M | 2 M | 1 M | 2 M |
| Description | Clear | Clear | Clear | Clear | Clear |
| pH | 7.66 | 7.06 | 6.8 | 7.54 | 7.43 |
| Assay | 101.7 | 99.2 | NA* | 100.2 | 101.7 |
| Preservative content | 96.2 | 98.8 | NA* | 99.8 | 96.2 |
| Related Substances | | | | | |
| Highest Unknown | 0.05 | 0.14 | 0.42 | 0.03 | 0.07 |
| Total impurities | 0.07 | 0.24 | 0.57 | 0.03 | 0.09 |

*NA = Not Available

The Composition C is physically and chemically stable for at least 2 months, without visible particles and with no significant change in assay and impurities.

### Example 3

TABLE 6:

| Ingredients | Composition (mg/mL) | |
|---|---|---|
| | F | G |
| Hydroxyurea | 100.0 | 100.0 |
| Glycine | 5.7 | 5.7 |
| Methyl paraben | 0.5 | 0.5 |
| Sucralose | 2.0 | 2.0 |
| Xanthan gum | 4.0 | 4.0 |
| L-Cysteine HCl monohydrate | -- | 0.5 |
| Disodium EDTA | 3.5 | 3.5 |
| Mixed berry flavor | 0.5 | 0.5 |
| Purified water | q.s | q.s |
| Final pH | 5.05 | 4.31 |

### Manufacturing Procedure of Compositions F and G:

Purified water (70% of the total quantity) was heated at 80° C. and methyl paraben was added and stirred continuously to get a clear solution. The solution was cooled to room temperature and sweetener, followed by flavor was added and stirred to get a clear solution. Hydroxyurea was added to the solution under continuous stirring at room temperature to get a clear solution. Further, Xanthan Gum, followed by glycine, L-cysteine HCI monohydrate, and disodium EDTA were added to above solution and stirred continuously at room temperature to get a clear solution. Purified water was added to make up the final volume. The solution was filtered using 5µ PVDF membrane filter and final filtered solution was filled in Amber Type I glass bottles or HDPE containers. Stability of composition F and G was evaluated at 2-8° C. and at 25° C./60%RH conditions.

TABLE 7

Stability data for Composition F and G

| Stability Data | Composition F | | | Composition G | | |
|---|---|---|---|---|---|---|
| Pack | HDPE Bottle | | | | | |
| Stability condition | Initial | 25° C./60% RH | 2-8° C. | Initial | 25° C./60% RH | 2-8° C. |
| Station | | 1 M | 1 M | | 1 M | 1 M |
| Description | Clear | Clear | Clear | Clear | Clear | Clear |
| pH | 5.05 | 6.01 | 5.17 | 4.31 | 5.92 | 4.22 |
| Assay | 99.4 | NA* | | | | |
| Preservative content | 98.5 | | | | | |
| Related Substances | | | | | | |
| Highest Unknown | 0.03 | 0.12 | 0.04 | 0.03 | 0.13 | 0.04 |
| Total impurities | 0.05 | 0.04 | 0.06 | 0.05 | 0.45 | 0.06 |

*NA = Not Available

The Composition F and G both are physically and chemically stable for at least 1 month, without visible particles and with no significant change in impurities.

### Example 4

TABLE 8:

| Ingredients | Composition (mg/mL) | |
|---|---|---|
| | H | I |
| Hydroxyurea | 100.0 | 100.0 |
| Arginine | - | 1.35 |
| Glycine | 5.7 | 5.7 |
| Methyl paraben | 0.5 | 0.5 |
| Sucralose | 2.0 | 2.0 |
| Xanthan gum | 4.0 | - |
| L-Cysteine HCl monohydrate | 1.1 | - |
| Disodium EDTA | 3.5 | - |
| Tartaric acid | 2.0 | - |
| Mixed berry flavor | 0.5 | 0.5 |
| Purified water | q.s | q.s |
| Final pH | 3.25 | 8.37 |

Purified water was heated at 80° C. to dissolve methyl paraben. The solution was later cooled to room temperature and sweetener, followed by flavor was added and stirred to get a clear solution. Hydroxyurea was added to the solution under continuous stirring at room temperature to get a clear solution. Further, L-Arginine, L-cysteine, glycine and tartaric acid and disodium EDTA were added to above solution (as per the respective batches) and stirred continuously at room temperature to get a clear solution. Purified water was added to make up the final volume. The solution was filtered using 5µ PVDF membrane filter and final filtered solution was filled in Amber Type I glass bottles or HDPE containers. Stability of composition H and I was evaluated at 2-8° C. and at 25° C./60%RH conditions.

TABLE 9

Stability data for Composition H

| Stability condition and Pack | Initial | 25° C./60% RH-HDPE bottle | 2-8° C.-HDPE bottle |
|---|---|---|---|
| Station | | 3 M | 3 M |
| Description | Clear | Clear | Clear |
| pH | 3.25 | 4.61 | 3.34 |
| Assay | 102.1 | 99.5 | 101.7 |
| Preservative content | 97.8 | 98.6 | 99.0 |
| Related Substances | | | |
| carbamoyloxyurea | ND* | 0.18 | 0.06 |
| Highest Unknown | 0.01 | 0.60 | 0.10 |
| Total impurities | 0.01 | 0.82 | 0.16 |

*ND =Not Detected

TABLE 10

Stability data for Composition I

| Stability condition and Pack | Initial | 25° C./60% RH-HDPE bottle | 2-8° C.-HDPE bottle |
|---|---|---|---|
| Station | | 3 M | 3 M |
| Description | Clear | Clear | Clear |
| pH | 8.37 | 6.93 | 7.71 |
| Assay | 100.0 | 96.8 | 99.1 |
| Preservative content | 98.6 | 92.7 | 95.0 |
| Related Substances | | | |
| carbamoyloxyurea | ND* | 0.43 | 0.17 |
| Highest Unknown | 0.03 | 0.04 | 0.05 |
| Total impurities | 0.04 | 0.47 | 0.22 |

*ND =Not Detected

The Composition H and I were both physically and chemically stable for at least 3 month, without visible particles and with no significant change in impurities.

1. A pharmaceutical composition comprising:
(i) hydroxyurea at a concentration of about 10 mg/mL to about 500 mg/mL;
(ii) at least one pharmaceutically acceptable liquid vehicle; and
(iii) one or more amino acids selected from the group consisting of glycine, alanine, glutamic acid, L-arginine, lysine, L-cysteine, methionine and mixtures thereof,
wherein the composition is in the form of a solution suitable for oral administration, and
wherein the solution is stable, such that the level of carbamoyloxyurea in the solution is less than 0.5% w/w as measured by HPLC, when the solution is stored at 25° C./60% RH for at least 3 months.

2. The composition according to claim 1, wherein hydroxyurea is present at a concentration of about 100 mg/mL.

3. The composition according to claim 1, wherein the solution is stable, such that the level of carbamoyloxyurea in the solution is less than 0.5% w/w as measured by HPLC, when the solution is stored at 2-8° C. for at least 12 months.

4. The composition according to claim 1, wherein the amount of amino acids is in the range of 0.1% to 10.0% based on the total weight of the composition.

5. The composition according to claim 1, wherein the concentration of amino acids is in the range of 0.1 mg/mL to 10.0 mg/mL.

6. The composition according to claim 1, wherein the solution is free of sodium hydroxide, potassium hydroxide, sodium bicarbonate, or sodium carbonate as pH adjusters or pH modifiers.

7. The composition according to claim 1, wherein the solution resists pH change such that the pH remains within a range of 4.0 to 9.0.

8. The composition according to claim 7, wherein the pH remains within a range of 5.0 to 8.0.

9. The composition according to claim 7, wherein the pH remains within a range of 6.0 to 7.0.

10. The composition according to claim 7, wherein the pH remains within a range of 5.0 to 6.0.

11. The composition according to claim 7, wherein the pH remains within a range of 4.0 to 6.0.

12. The composition according to claim 7, wherein the pH remains within a range of 7.0 to 9.0.

13. The composition according to claim 7, wherein the pH remains within a range of 7.0 to 8.0.

14. The composition according to claim 1, wherein the pharmaceutically acceptable liquid vehicle is selected from group consisting of water, glycerin, alcohols, propylene glycol, polyethylene glycol, and mixtures thereof.

15. The composition according to claim 1, wherein the solution further comprises one or more pharmaceutically acceptable excipients selected from the group consisting of stabilizers, solubilizers, pH modifiers, pH adjusting agents, buffering agents, preservatives, thickening agents, antioxidants, chelating agents, flavoring agents, sweetening agents, coloring agents and mixtures thereof.

16. The composition according to claim 1, wherein the solution is free of preservatives.

17. A pharmaceutical composition comprising:
(i) hydroxyurea at a concentration of about 10 mg/mL to about 500 mg/mL; and
(ii) at least one pharmaceutically acceptable liquid vehicle, wherein the composition is in the form of a solution suitable for oral administration and wherein the solution is free of sodium hydroxide, potassium hydroxide, sodium bicarbonate, or sodium carbonate as pH adjusters or pH modifiers; and wherein the solution is stable, such that the level of carbamoyloxyurea in the solution is less than 0.5 % w/w as measured by HPLC, when the solution is stored at 25° C./60% RH for at least 3 months or 2-8° C. for at least 12 months.

18. A pharmaceutical composition comprising:
(i) hydroxyurea at a concentration of about 10 mg/mL to about 500 mg/mL; and
(ii) at least one pharmaceutically acceptable liquid vehicle,
wherein the composition is in the form of a solution suitable for oral administration, and
wherein the solution resists pH change such that the pH remains within a range of 4.0 to 6.0 or 7.0 to 9.0 when stored at 25° C./60% RH for at least 3 months or at 2-8° C. for at least 12 months.

19. The composition according to claim 18, wherein the solution is stable, such that the level of carbamoyloxyurea in the solution is less than 0.5 % w/w as measured by HPLC, when the solution is stored at 25° C./60% RH for at least 3 months or 2-8° C. for at least 12 months.

20. A method of reducing the frequency of painful crises and/or reducing the need for blood transfusions in pediatric or adult patients with sickle cell anemia with recurrent moderate to severe painful crises comprising administering the composition according to claim 1 to the patient.
